(19) United States
(12) Patent Application Publication
(10) Pub. No.: US 2012/0296191 A1
(43) Pub. Date: Nov. 22, 2012

(54) TRANSDUCER MOUNTINGS AND WEARABLE MONITORS

(76) Inventors: Matthew John Ross McGrath, Edinburgh (GB); Erik Weber Jensen, Barcelona (ES)

(21) Appl. No.: 13/502,148

(22) PCT Filed: Oct. 15, 2010
(86) PCT No.: PCT/GB2010/051743

§ 371 Date: Aug. 1, 2012

(30) Foreign Application Priority Data
Oct. 16, 2009 (GB) 0918157.9
Oct. 16, 2009 (GB) 0918159.5

(51) Int. Cl. A61B 5/0478

(52) U.S. Cl. 600/383

(57) ABSTRACT

A transducer mounting comprising a compliant elongate support member having a skin engaging surface, adhering means to adhere the skin engaging surface to a wearer's head, and a plurality of transducers longitudinally spaced on the skin engaging surface. A wearable monitor comprising adhering means for adhering the wearable monitor to skin, a skin-facing surface with a transducer thereon for either or both stimulating a physiological response and measuring a physiological parameter, and display means or audio output means integral to the wearable monitor for displaying a signal related to a measured physiological parameter on the wearable monitor or transmitting a sound related to a measured physiological parameter from the wearable monitor, respectively.

### FIELD OF THE INVENTION

The invention relates to transducer mountings and wearable monitors, such as transducer mountings for attaching electrodes, and possibly one or more sensors or stimulators, to a wearer's skin, and wearable monitors for monitoring one or more physiological parameters.

### BACKGROUND TO THE INVENTION

Some aspects of the invention concern transducer mountings including electrodes or other sensors for mounting to a wearer's body, including but not limited to devices adapted for mounting to a wearer's head. Transducer mountings and wearable monitors may be used to measure physiological signals, for example, electroencephalogram (EEG) signals. In this case, the wearer may, for example, be a patient, a subject or a user.

One application for transducer mountings is to mount transducers including sensors for the measurement of one or more parameters concerning anaesthesia in a patient during a medical procedure, for example, a measurement of consciousness, nociception or muscle relaxation.

It is advantageous to provide a transducer mounting including a plurality of transducers (for example, a headset including a plurality of electrodes for monitoring EEG signals). It can be easier to attach a single mounting to a patient than several separate transducers. Wires will typically extend through the transducer mounting and so it is easier to attach a single transducer mounting to a patient, with a single wired connection, then several separate transducers each of which has a separate wired connection.

It is known to provide transducer mountings in the form of electrode mountings comprising a broad, thin, elongate adhesive sheet with a plurality of electrodes on a head engaging surface of the electrode mounting. The electrodes are surrounded entirely by adhesive and the resulting electrode mountings occupy a considerable surface area of a patient's head.

However, electrode mountings of this type present several practical problems. Firstly, it can be difficult to shape a broad, thin, elongate adhesive sheet to the contours of an individual patient's head. If a thin sheet is flexed significantly around an axis normal to the sheet, it will crease. The resulting creases may get snagged. A broad expanse of adhesive can be difficult to position accurately so that the electrodes are in a desired location. Accordingly, some aspects address the problem of providing a mounting for electrodes and/or other transducers which can be better fitted to the contours of an individual patient's head.

Furthermore, conventional electrode mountings occupy a substantial surface area on a patient's head. This is undesirable as this occupies surface area which might otherwise be useful for other types of monitoring, for example, with other sensors, or by simple visual inspection. This can be especially problematic during operations on the head, for example, brain surgery. Accordingly, some aspects address the problem of reducing the surface area occupied by mountings for electrodes and/or other transducers.

Further aspects of the invention address problems concerning wearable monitors for monitoring physiological parameters and issues concerning the display of measured physiological parameters including, but not limited to, the display of measured physiological parameters concerning anaesthesia in a patient during an operation.

### SUMMARY OF THE INVENTION

According to a first aspect of the present invention there is provided a wearable monitor comprising one or more fixing elements for fixing the wearable monitor to a body (generally the body of a mammal, typically a human being), a body-facing surface and a transducer for either or both stimulating a physiological response and measuring a physiological parameter, and display means or audio output means integral to the wearable monitor for displaying a signal related to a measured physiological parameter on the wearable monitor or transmitting a sound related to a measured physiological parameter from the wearable monitor, respectively.

It may be that the display means or audio output means are audio output means (for typically, a loudspeaker) which transmit a sound related to a measured physiological parameter. For example, an alarm sound may be generated by the audio output means responsive to a measured physiological parameter meeting a predetermined alarm condition. The alarm sound should be audible from at least 1 meter away by a person with normal hearing.

The display means or audio output means may be display means for displaying a signal or transmitting a sound related to a measured physiological parameter on the wearable monitor. The display means may be an electronic image display, such as an LED, LCD or electronic paper screen. Thus, the invention extends to a wearable monitor comprising one or more fixing elements fixing for fixing the wearable monitor to a body, a body-facing surface with a transducer thereon for either or both stimulating a physiological response and measuring a physiological parameter, and integral display means for displaying a signal related to a measured physiological parameter on the wearable monitor.

By providing display means which are integral to the wearable monitor, a user (for example a medical practitioner such as a doctor or a nurse during an operation in which the wearable monitor is fixed to the body of a patient) may view data concerning the measured physiological parameter simply by looking at the wearer. During operations, this can save time and enable medical practitioners to concentrate their attention on the patient.

Typically, the body-facing surface has the transducer thereon. However, it may be that the transducer is within the wearable monitor. For example, the transducer may be contactless capactive sensing electrode.

The one or more fixing elements may be adhesion means to adhere the wearable monitor to the body. The one or more fixing elements may comprise a band such as a head band or wrist band, a head set or one or more clips. One or more fixing elements may comprise a connector for connecting the wearable monitor to a band.

Thus, the invention extends to a wearable monitor comprising adhesion means for adhering the wearable monitor to skin, a skin-facing surface with a transducer thereon for either or both stimulating a physiological response and measuring a physiological parameter, and display means or audio output means integral to the wearable monitor for displaying a signal related to a measured physiological parameter on the wearable monitor or transmitting a sound related to a measured physiological parameter from the wearable monitor, respectively.

It may be that the physiological parameter is a parameter specific to a part of the body to which the wearable monitor can be attached. For example, the wearable monitor may be a head mountable sensor for monitoring one or more parameters concerning anaesthesia.

The wearable monitor may be a wearable sensor and the transducer may be a sensor. The sensor may be an electrode. However, the sensor may be an acoustic sensor, optical sensor, chemical sensor, non-contact capacitive sensor or other type of sensor. The wearable monitor may be an actuator and the transducer may be an actuator such as an acoustic generator, for example an ultrasound generator, a surface acoustic wave device, or a loudspeaker, or a light source. The actuator may comprise one or more electrodes, for example, to generate electrical stimuli to a wearer. However, the transducer may function as both a sensor and an actuator.

Preferably, the display means do not protrude significantly from the wearable monitor. Preferably, the wearable monitor has a body engaging surface and an opposite outer surface. Preferably, the display means are generally flush with the outer surface. Thus, a low profile display is provided. Preferably, the wearable monitor has a depth to length (measured along its major dimension) ratio of less than 0.5, or preferably less than 0.2 or 0.1.

Where the one or more fixing elements are adhesion means, the adhesion means are typically provided on the body-facing surface. The body-facing surface may be a skin-facing surface.

The display means may display a visual signal dependent on the quality of the electrical connection between an electrode and body. An electrical connection quality signal may simply be a light source (such as a solid state light emitting diode or an organic light emitting polymer) which glows to indicate that the electrical connection has a sufficiently low resistance. Where the wearable monitor includes one or more electrodes, a visual indicator operable to display a visual indication dependent on the quality of the electrical connection between an electrode and body is typically located on an outer surface of the wearable monitor, directly over the respective electrode. Where the wearable monitor is or comprises a transducer mounting according to the first aspect of the invention a respective visual indicator is typically provided on the elongate support member opposite some or all said electrodes, operable to display a visual indication dependent on the quality of the electrical connection between the respective electrode and body. By providing a plurality of visual indicators, at least one associated with each of at least two electrodes located along the length of a compliant elongate support member, each of which is operable to display a visual indication dependent on the quality of the electrical connection between the respective electrode and the body, the fitting of the transducer mounting is facilitated, as each electrode may be positioned in turn and checked for the quality of its electrical connection before the next electrode along the elongate support member is adhered.

The display means may comprise one or more light sources operable to display a visual signal related to a measured physiological parameter by the intensity or colour of light emitted by the light source. Thus, the visual signal may simply comprise the intensity and/or colour of emitted light. The display means may comprise a plurality of light sources operable to display a visual signal related to a measured physiological parameter by the intensity or colour of light emitted by number of light sources which emit light, or which emit light of a particular intensity or colour.

The display means may be operable to display an image including numbers or text related to a measured physiological parameter. The display means may be operable to display an image including shapes selected depending on the measured physiological parameter. The display means may comprise a two dimensional array of pixels, for example, the display means may comprise an LED (e.g. OLED) or LCD display screen. The display means may be touch sensitive. Thus, the display means may be operable to change a or the parameter which is displayed, or how it is displayed, responsive to touch or to a specific touch stimulus.

The display means may have a display area of at least 3 cm², preferably at least 4cm² and more preferably at least 8 cm². The display means may have a display area of at least 12 cm². A relatively large display means enables the data shown on the display to be viewed remotely, allowing patient monitoring from a distance.

The display means may be operable to display visual signals related to more than one physiological parameter. For example, the display means may display first and second visual indications, each of which is related to a different physiological parameter.

The visual signals may be a chart. The visual signals may show time varying data. The visual signals may show historic data, e.g. a measured time course. The visual signals may show predicted data.

The wearable monitor may comprise one or more electronic interfaces, which may be wired or wireless, for electronic communication with external data processing apparatus. The wearable monitor may comprise an integral power source, for example a battery. The wearable monitor may comprise a processor. The processor may be operable to compute the quality of the electrical connection between one or more said electrodes and a wearer. The processor may be operable to calculate a physiological parameter from measurements conducted using the one or more said electrodes.

The wearable monitor may be operable to communicate a measured signal to a remote processor, for example by way of a wired or wireless connection, and to receive a physiological parameter calculated taking into account the communicated measured signal, and the display means may display a visual indication related to the received calculated physiological parameter. This can be especially useful where the calculations required to determine the physiological parameter are complex or also require input from sensors which are not part of the wearable monitor. For example, the communicated measured signal may be an electrical signal received at one or more electrodes and the received physiological parameter may be a parameter related to anaesthesia, for example related to one or more of consciousness, nociception and muscle relaxation.

The wearable monitor may be adapted for being fixed to one or more of an arm, a leg, a face, a chest, or the neck (e.g. adam's apple). The physiological parameter may be respiratory patterns, particularly where the wearable monitor is adapted for being fixed to the chest or neck.

The wearable monitor may comprise a body portion and a retaining portion (e.g. an envelope portion), the retaining portion demountably retaining (e.g. demountably enveloping) the body portion; the body portion comprising the display means or audio output means for displaying a signal related to a measured physiological parameter on the wearable monitor or transmitting a sound related to a measured physiological parameter from the wearable monitor, respectively; the retaining portion comprising the one or more fixing elements to fix the wearable monitor to a body (generally the body of a mammal, typically a human being), and a transducer for either or both stimulating a physiological response and measuring a physiological parameter. The body portion may be demountably retained by (typically within) the retaining portion. The body portion may be reusable. The retaining portion may be disposable. Thus, the body portion may be removed from the retaining portion and later mounted onto (typically into) another retaining portion. Thus the part which contacts a patient is disposable. The body portion and the retaining portion may comprise electrical contacts to enable the body portion to control or receive signals from transducers in the retaining portion. The body-facing surface typically has the transducer thereon. The body-facing surface is typically a skin-facing surface.

The invention also extends in a second aspect to a kit for a wearable monitoring, the kit comprising a body portion and a retaining portion (e.g. an envelope portion), the retaining portion demountably retaining (typically demountably enveloping) the body portion; the body portion comprising the display means or audio output means for displaying a signal related to a measured physiological parameter on the wearable monitor or transmitting a sound related to a measured physiological parameter from the wearable monitor, respectively; the retaining portion comprising the one or more fixing elements to fix the wearable monitor to a body (generally the body of a mammal, typically a human being), and a transducer for either or both stimulating a physiological response and measuring a physiological parameter. Further optional feature correspond to those discussed above in relation to the first aspect.

The invention extends in a third aspect to a wearable transducer mounting comprising a compliant elongate support member having a body engaging surface (typically a skin engaging surface), one or more fixing elements to fix the body engaging surface to a wearer's body (typically the wearer's head, the wearer typically being a mammal, e.g. a human being), and a plurality of transducers longitudinally spaced along the compliant elongate support member.

By a transducer we include a sensor or an actuator. The or each transducer may be an electrode. The transducer mounting may be an electrode mounting. The transducer mounting may be for mounting transducers to a wearer's head and, in this case, the body engaging surface is be a head engaging surface. The body-engaging surface may be a skin engaging surface. The adhering means may be configured to adhere the wearable transducer mounting to skin.

Typically, the plurality of transducers are longitudinally spaced along the body-engaging (typically skin engaging) surface. However, the transducers may not require to contact the wearer's body. For example, they may be contactless capacitive sensors. Where the transducers do not require to contact the wearer's body, the body-engaging surface (e.g. skin engaging surface) may contact the wearer's body (e.g. skin) only at spaced apart locations along the body-engaging surface (e.g. skin engaging surface). Otherwise, the body-engaging surface is typically configured to contact the wearer's body (typically head, for example, skin) continuously along at least the majority (and optionally all) of the length of the compliant elongate support member.

The one or more fixing elements may be adhering means to adhere the wearable monitor to the body. The one or more fixing elements may comprise a band such as a head band or wrist band, a head set or one or more clips. One or more fixing elements may comprise a connector for connecting the wearable monitor to a band.

The invention extends to a wearable transducer mounting comprising a compliant elongate support member having a skin engaging surface, adhering means to adhere the skin engaging surface to a wearer's head, and a plurality of transducers longitudinally spaced on the skin engaging surface.

A compliant elongate support member may be readily fitted to the contours of an individual wearer's body, for example to the shape of their head. Typically, where the compliant elongate support member comprises one or more adhering means, the adhering means extend along at least the majority of the length of the elongate support member. The adhering means may extend along the least majority of the length of the skin engaging surface which is not occupied by the plurality of transducers.

The adhering means may comprise one or more adhesive strips. The or each adhesive strip may be covered with a release strip. It may be that the adhering means (for example, one or more adhesive strips) extend longitudinally on either side of each transducer, but do not extend laterally of each transducer. This enables the elongate support member to be less broad (have less lateral extent) than if adhering means extended around the entire periphery of each transducer. Accordingly, a transducer mounting may be provided which occupies a smaller surface area of a wearer's body (for example, their head) than would otherwise be the case. The minimisation of surface area is especially helpful for mounting transducers to the head, or, for example, to the hand, or to any part of neonatal or very young patients.

By longitudinally, we refer to a direction along the length of the elongate support member. By laterally, we refer to a direction perpendicular to the length of the elongate support member, and generally parallel to a wearer's skin in use.

Each transducer (e.g. electrode) may have a breadth of at least 75%, and preferably at least 90% of the breadth of the or each skin engaging surface where the respective transducer (e.g. electrode) is mounted.

Preferably, the breadth of the elongate support member in the region of each transducer (e.g. electrode) and/or adjacent and intermediate each transducer and/or along the majority of the length of the elongate support member, at least including the regions adjacent each transducer, and/or along the entire length of the elongate support member, is less than 20 mm, less than 10 mm, or preferably less than 8 mm.

Preferably, the breadth of the elongate support member in the region of each transducer (e.g. electrode) and/or adjacent and intermediate each transducer and/or along the majority of the length of the elongate support member, at least including the regions adjacent each transducer, and/or along the entire length of the elongate support member, is at least 2 mm, or preferably at least 4 mm.

Preferably, the elongate support member has a length which is greater than 10 times its maximum breadth.

Transducer mountings having elongate support members of such dimensions can be adhered to a wearer's head, while minimising the space occupied by each transducer mounting. This may be especially important where the wearer is a patient under anaesthesia, particular a patient undergoing surgery on the head, such as brain surgery. Space saving is also particularly important when operating on neonatal and infant patients.

Preferably, the depth of the elongate support member in the region of each transducer (e.g. electrode) and/or adjacent and intermediate each transducer and/or along the majority of the length of the elongate support member, at least including the regions adjacent each transducer, and/or along the entire length of the elongate support member, is at least 2 mm, or preferably at least 4 mm, although it may be 10 mm or more. This enables the transducer mounting to be readily grasped by a user, by pinching the strip between their fingers. This can substantially facilitate placement of the transducer mounting on a patient.

In addition to the body engaging (e.g. skin engaging) surface, the elongate support member may therefore comprise at least two longitudinally extending surfaces at an angle of at least 60 degrees to each other which do not have adhesive on them. This enables the elongate support member to be pinched without contact with adhesive and contrasts with broad, planar sheets having one adhesive surface and only one adhesive free surface (the opposite surface) of significant extent, which cannot be pinched.

To facilitate pinching, the elongate support member may have a breadth to thickness ratio of between 0.25 and 4.0, preferably between 0.5 and 2.0 and most preferably between 0.75 and 1.50, at least in the region of each transducer (e.g. electrode) and/or adjacent and intermediate each transducer and/or along the majority of the length of the elongate support member, at least including the regions adjacent each transducer, and/or along the entire length of the elongate support member.

The adhering means may comprise one or more compressible chambers provided within the elongate support member and at least one aperture extending from each compressible chamber through the elongate support member to the body engaging (typically skin engaging) surface. Typically, the or each compressible chamber is airtight except for one or more respective apertures. Thus, the elongate support member may be adhered to a patient's body (e.g. head) by compressing the one or more compressible chambers, for example by pinching, and attaching the body engaging (typically skin engaging) surface to a patient's body (e.g. head), so that the resulting low-pressure within the compressible chambers at here is the transducer mounting to the patient's body (e.g. head) by way of suction. One or more adhesive members may be provided in addition to one or more compressible chambers.

Accordingly, the transducer mounting may be conveniently positioned on a patient's body (e.g. head), for example across the forehead, and adhered to the patient's body (e.g. head) by pinching the elongate support member, flexing the elongate support member to the desired configuration, bringing it into contact with the patient's body (e.g. head), and letting it go, whereupon it is retained at least in part by suction.

The elongate support member is preferably formed from a compliant material. The elongate support member may be formed from a pliable material capable of retaining a shape to which it is deformed. The elongate support member may be resilient.

The elongate support member is preferably flexible around an axis normal to the body engaging surface, with a radius of curvature of less than 10 cm and preferably less than 5 cm or less than 2.5 cm, without buckling. Thus, the transducer mounting may be conveniently shaped to a patient's head, without buckling. Another advantage of providing an elongate support member with a breadth to thickness ratio of between 0.25 and 4.0, preferably between 0.5 and 2.0 and most preferably between 0.75 and 1.50, at least in the region of each transducer (e.g. electrode) and/or adjacent and/or adjacent and intermediate each transducer and/or along the majority of the length of the transducer support member is that this facilitates flexing around an axis normal to the body engaging surface without buckling. This contrasts with known thin, broad sheets which buckle readily if flexed significantly around an axis normal to the body engaging surface.

The elongate support member may have a three sided cross-section along the majority of its length. The elongate support member may be generally trigonal or triangular in cross section. The elongate support member may be a generally triangular prism. The triangle may be generally isosceles, for example generally equilateral.

The elongate support member may comprise one or more raised portion which extends further from the body engaging surface than surrounding regions of the elongate support member. Raised portions may be spaced apart. A raised portion may have a depth of 2 mm to 10 mm more than adjacent (e.g. intermediate) regions of the elongate support member. The provision of one or more raised portions facilitates manual handling of the elongate support member when the elongate support member is fitted to a wearer's head.

The transducer mounting may comprise at least three electrodes on the body engaging (typically skin engaging) surface of the elongate support member. Thus, the transducer mounting may be a head mountable transducer mounting for monitoring EEG signals, for example, to monitor one or more anaesthesia related parameters concerning a wearer. A depth of anaesthesia related parameter may, for example, be awareness level, nociception or muscle relaxation. An electrical circuit may be provided for providing an electrical stimulus using one or more transducers which are electrodes, for example, to generate a measurable reaction.

Where the transducers are electrodes, the transducer mounting may be formed to retain at least two electrodes on a wearer's forehead, above the eyeline, and a third electrode on a wearer's cheek, below the eyeline, preferably without buckling. Thus, the transducer mounting may be sufficiently inherently curved or sufficiently flexible in a zone intermediate the said at least two electrode and the said third electrode to curve between a wearer's forehead and their cheek, either in front of, over, or behind the ears without buckling.

The transducer mounting may be adapted to extend at least in part around the back of a patient's head. The transducer mounting may be adapted to extend over the top of a patient's head.

Alternatively, the transducer mounting may comprise a connecting member, a first compliant elongate support member extending from the connecting member having a body engaging surface, adhering means to adhere the body engaging surface to a wearer's body (e.g. head), and a plurality of transducers (e.g. electrodes) on the skin engaging surface of the first compliant elongate support, and a second compliant elongate support member extending from the connecting member and having a body engaging surface, adhering means to adhere the body engaging surface to a wearer's body (e.g. head), and at least one transducer (e.g. electrode) on the body engaging surface of the second compliant elongate support, wherein the first and second compliant elongate support members extend from the connecting member at spaced apart locations. The connecting member may be a resilient or rigid curved member extending between the first and second compliant elongate support members.

The provision of a transducer mounting comprising electrodes for attachment to the forehead, above the eyeline, and at least one electrode for attachment to the cheek, below the eyeline, facilitates convenient application and retention of electrodes at these separate locations. However, the elongate support member may comprise three or more electrodes for location above the eyeline.

It may be that the transducer mounting is formed to retain at least two electrodes above a patient's eyeline and at least two electrodes below the patient's eyeline to facilitate use of the transducer mounting on the left or right side of a patient. The transducer mounting may have bilateral symmetry to facilitate use on the left of right side of a patient.

The connecting member may comprise a sound generation module for generating sounds in a wearer's ear or a fitment for demountably attaching a sound generation module to the transducer mounting. The fitment may enable the sound generation module to be retained in either of two opposite orientations to facilitate location of the transducer on either side of a patient's body, for example either side of a patient's head. A sound generation module, such as a loudspeaker, may be used to generate auditory evoked potentials in a wearer. In this case, the transducers may be electrodes used to measure the evoked potentials. The sound generation module may comprise a loudspeaker configured to extend into a wearer's aural cavity. Alternatively, the sound generation module may comprise a loudspeaker and a mounting for locating the loudspeaker in contact with a wearer's head to transmit sound to a wearer's cochlea by conduction through the skull. In the latter case, the sound generation module is preferably adapted for mounting in contact with a wearer's head, adjacent an ear, without obscuring the earhole. This enables a wearer (e.g. a patient) to better hear ambient sounds.

The transducer mounting may comprise one or more interfaces, which may be wired or wireless, for electronic communication with external data processing apparatus. The transducer mounting may comprise an integral power source, for example a battery. The transducer mounting may comprise a processor. The processor may be operable to compute the quality of the electrical connection between one or more transducers (typically electrodes) and a wearer. The processor may be operable to calculate a physiological parameter from measurements conducted using the one or more said transducers (e.g. electrodes).

The length of the elongate support member may be adjustable. For example, the elongate support member may comprise an elastic portion, two portions which are longitudinally slidable relative to each other, a flexible concertina portion, a collapsible portion, a demountable portion. The elongate support member may be adjustably mounted to a connecting portion or housing to enable the distance from which the elongate support member extends from the connection portion or housing to be varied. The connecting portion or housing may be for location at, around or near a patient's ear and may comprise a loudspeaker for generating AEPs. For example, the elongate support member and connecting portion or housing may be slidably mounted relative to each other. They may have cooperating formations demountably engageable in more than one longitudinal position relative to each other. The elongate support member may extending from a housing and be retractable at least in part into the housing. The housing may comprise a loudspeaker and the location of the loudspeaker may be moveable relative to the elongate support member. The attachment between the connecting portion or housing and the elongate support member may be reversible so that the connecting portion or housing may be demountably attached to the elongate support member in either of two opposite orientations, facilitating provision of a transducer mounting which is attachable to either side of a patient, for example to either the left or right side of their head.

The transducer mounting may comprise a body portion and a retaining portion (e.g. an enveloping portion) demountably retaining (e.g. demountably enveloping) the body portion; the retaining portion comprising the compliant elongate support member, adhering means and a plurality of transducers longitudinally spaced on the body engaging surface, the body portion comprise a display means or audio output means for displaying a signal or transmitting a sound related to a physiological parameter measured by the wearable transducer. The body portion may be demountably retained by (e.g. within) the retaining portion. The body portion may be reusable. The retaining portion may be disposable. Thus, the body portion may be removed from the retaining portion and later mounted into another retaining portion. Thus the part which contacts a patient is disposable. The body portion and the retaining portion may comprise electrical contacts to enable the body portion to control or receive signals from transducers in the retaining portion. The body engaging surface may be a skin engaging surface.

The invention also extends in a fourth aspect to a kit for a transducer mounting, the kit comprising a body portion and a retaining portion (e.g. an enveloping portion) configured to demountably retain (e.g. demountably envelop) the body portion; the retaining portion comprising the compliant elongate support member, adhering means and a plurality of transducers longitudinally spaced on the body engaging surface, the body portion comprise a display means or audio output means for displaying a signal or transmitting a sound related to a physiological parameter measured by the wearable transducer. Further optional features correspond to those discussed above in relation to the third aspect.

### DESCRIPTION OF THE DRAWINGS

An example embodiment of the invention will now be illustrated with reference to the following Figures in which:

FIG. 1 is a perspective view of a headset, from the head-engaging side;

FIGS. 2A and 2B illustrate the outward-facing side of a headset, adhered to a wearer, from two different angles;

FIGS. 3A and 3B provide corresponding images of an alternative headset;

FIGS. 4A and 4B illustrate an embodiment of the invention employing two separate electrode mountings;

FIG. 5 is a perspective view of a compliant elongate support member;

FIG. 6A through 6E are cross-sections through alternative compliant elongate support members;

FIG. 7 illustrates an ear-mountable transducer, demountably attachable to a headset;

FIG. 8 is a side view of a headset incorporating the ear-mountable transducer of FIG. 7;

FIG. 9 illustrates an alternative numerical display;

FIG. 10 is a plan view from above of a wearable monitor in the form of a patch; and

FIG. 11 is a plan view of the underside of the wearable monitor of FIG. 10.

### DETAILED DESCRIPTION OF AN EXAMPLE EMBODIMENT

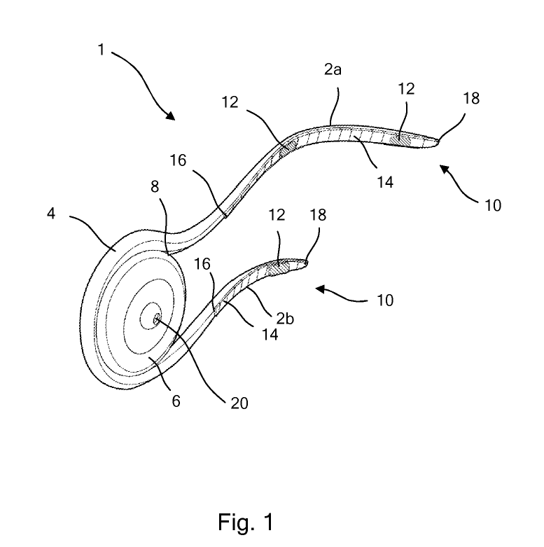

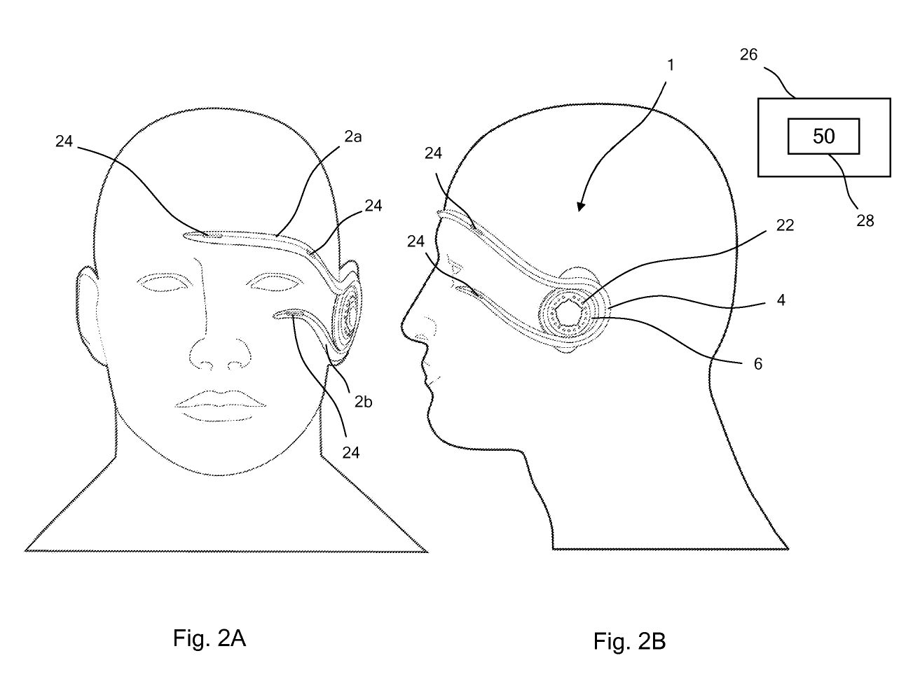

With reference to FIGS. 1 through 3, a headset 1 (functioning as a transducer mounting and as a wearable monitor) comprises first and second flexible arms 2a, 2b. The first and second flexible arms may each function as a compliant elongate support member or, in embodiment where they are continuous, may together form a compliant elongate support member.

The first and second flexible arms are formed integrally with a connecting portion 4 extending around and retaining an earpiece 6 in a groove 8. The first and second arms are formed form a compliant material and each has a head-engaging surface 10. The head-engaging surface of the first flexible arm has two longitudinally spaced apart elongate electrodes 12 and the head-engaging surface of the second flexible arm has one elongate electrode. The arms typically extend for 5 to 10 cm from the connection portion. In a first example configuration, they have a breadth of about 5 mm and a depth of about 5 mm along the majority of their length.

The first and second flexible arms have strips of contact adhesive 14 on the head-engaging surface along the majority of their length, from a proximal region 16 to the respective distal tip 18, except that the contact adhesive is interrupted by the electrodes. The contact adhesive does not extend laterally of each electrode. This enables the first and second flexible arms to be significantly narrower than would be the case if the adhesive extended around the entire periphery of each electrode. The contact adhesive may be covered by a release lining (not shown) which can be removed before use. Electrodes may be covered by a conductive gel (not shown), and a release lining may also extend across the conductive gel to preserve the gel before use.

The earpiece includes a loudspeaker 20, which extends into a wearer's ear hole, to generate sounds. Thus, the headset comprises arms which function as electrode mountings, and the headset also functions as a wearable monitor. The earpiece also includes a display screen 22 that is flush with the outer surface of the earpiece and which is discussed further below. On an outer surface of the elongate arms are provided LED light sources 24, which may, for example, be formed from flexible organic light emitting polymers, each of which is located so that, when the headset is attached to a wearer's head, each LED light source is located directly over a corresponding electrode.

The headset typically also includes one or more interfaces. For example, the headset may include a wired connection to enable remote monitoring apparatus 26 to make measurements of signals, such as EEG signals, using the electrodes of the headset. In this case, the remote monitoring apparatus may also provide power, through a wired connection, to power the LED light sources and display screen. The remote monitoring apparatus may include a screen 28 to display a physiological parameter monitored using the headset. However, preferably, the headset includes an internal power supply, such as a battery, and a wireless communications interface to transmit measured data and, in some embodiments, receive data concerning calculated physiological measurements, from the remote monitoring apparatus. In some embodiments, the headset includes an internal power supply and a processor capable of calculating a physiological measurement using the electrodes, and any other sensor which is present, and indicating this using LED light sources and the display screen, without reliance on any remote monitoring apparatus. However, even in this case, where the headset can function as a stand-alone device, the headset may include a wireless interface for communicating physiological measurements to optional remote monitoring apparatus.

An example application of the headset to the monitoring of an EEG signal, to measure the level of consciousness of a patient during an operation in which they will be anaesthetised, will now be described. However, the headset may be used in other contexts. For example, to monitor tiredness in a person operating a machine, such as industrial plant, or a vehicle.

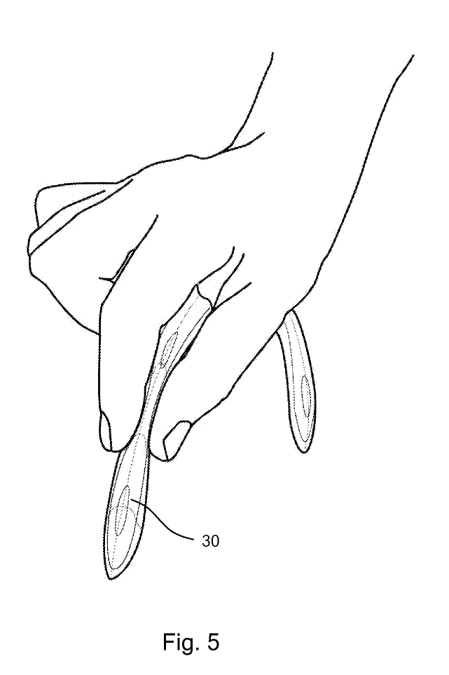

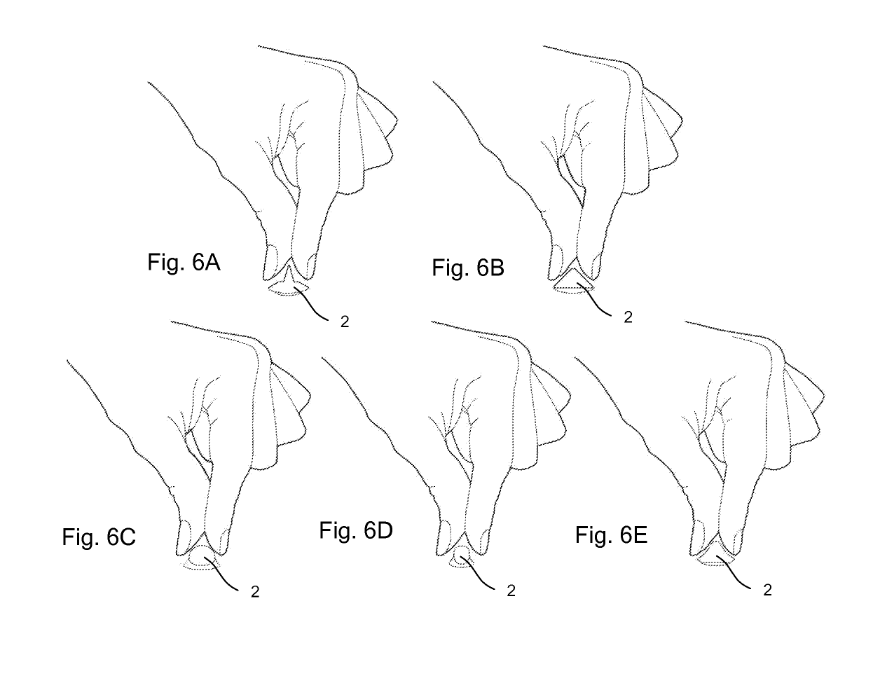

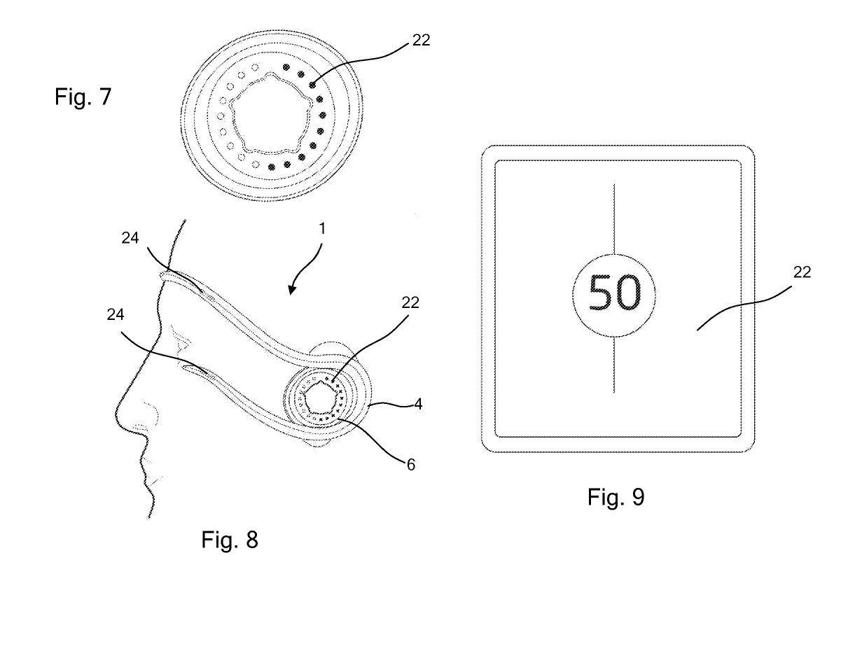

In order to attach the headset to a patient's head, the release paper is removed from at least some of the adhesive strips. A user, for example in medical practitioner preparing a patient for an operation, brings one part of one of the arms, into contact with the patient. They may start by locating a single electrode at a desired location on the patient's head. Alternatively, they may start by locating one end of one of the arms, such as the distal tip, or a proximal region. As illustrated in FIGS. 5 and 6A through 6E, the arm may be pinched between a user's thumb and index finger, enabling a user to conveniently flex the arms. A user might also flex a portion of the arm by pinching one part between the thumb and index finger of one hand, and pinching another part, a few centimetres away, between the thumb and index finger of their other hand, and then moving their hands to flex the arm. Thus, a user can bend the arm so that it adopts a desired shape on the patient's head, taking into account the unique size and shape of their anatomical features. The adhesive may be pressed into contact with the patient's head as the user progresses. They might complete attachment of one arm before separately attaching the other arm to a patient. The earpiece can then be mounted within the connecting portion. Alternatively, the earpiece may be already attached to the headset, and the user might locate the loudspeaker within a patient's ear first, before beginning to attach the arms.

As each electrode is attached to the skin, the LED light source opposite the electrode glows to indicate the quality of the electrical connection between the electrode and the patient's skin. The quality of the electrical connection may be communicated by the intensity or colour of the light emitted by the respective LED light source. For example, a coloured light might be emitted when the electrode first makes contact with a patient's skin and a bright white light may be emitted when the quality of the electrical connection is sufficiently good (sufficiently low resistance). The various LED light sources may emit a further signal (e.g. change colour or intensity, or illuminate in a predetermined pattern) to indicate visually when all of the electrodes have formed suitable electrical connections. By providing a visual indication of the quality of electrical connection in situ, while the electrodes are being secured to the patient, and directly over the electrodes, a user can receive immediately feedback which helps them to rapidly and effectively fit the headset.

In an example application, the headset is attached to a remote monitoring interface by a wired connection. Electrical signals from each electrode are communicated to the remote monitoring interface through the wired connection. The remote monitoring interface uses the electrical signals from each electrode to analyse a patient's EEG to determine a parameter related to anaesthesia, such as level of consciousness (being an example of a physiological parameter). The resulting level of consciousness is displayed on the remote display and updated continuously.

A number of methods of analysing and EEG signals to determine a level of consciousness of a patient are known to one skilled in the art. For example, it is known to measure the bispectral power of the EEG signal and to display this as a level of consciousness, for example according to the BIS scale (BIS is a trade mark of Aspect Medical, Inc.). An alternative method of calculating a measurement of a level of awareness, using a cheek-mounted electrode to compensate for interference by an a facial electromyogram (EMG) signal is disclosed in WO2008/138340 (Morpheus Medical), the contents of which are incorporated herein by virtue of this reference.

As well as measurement of a level of consciousness by the processing of non-evoked EEG signals, the loudspeaker may be used to generate sounds in the patient's ear which in turn create auditory evoked potentials (AEPs) within the patient's brain which can be extracted from the EEG signals and analysed to determine a level of consciousness. Methods of generating AEPs and analysing the resulting signals to determine a level of consciousness are known, for example, from WO2001/074248 (Danmeter A/S) and Methods of Information in Medicine, (1996), vol. 35, pp. 256-260, “Autoregressive Modeling with Exogenous Input of Middle-Latency Auditory-Evoked Potentials to Measure Rapid Changes in Depth of Anesthesia” (E. W. Jensen et al), the contents of which are incorporated herein by virtue of this reference.

Furthermore, an indication of the calculated level of consciousness is also transmitted back to the headset through the wired connection and displayed on the screen which is integral to the earpiece. The indication could be a numerical value, however, the indication may be the colour or intensity of light emitted by one or more light sources. An indication could also be provided by way of a scale, for example the number of adjacent light sources, arranged around a circle, which are illuminated simultaneously may indicate the calculated level of consciousness.

By displaying the measured physiological parameter in situ, a medical practitioner, for example an anaesthetist, may view an indication of the physiological parameter while looking directly at the patient. In this case, the physiological parameter relates to brain function and can be viewed while the practitioner looks directly towards the patient's head. This avoids or reduces the need for the medical practitioner to direct their line of site away to a separate monitor and enables a practitioner to stay more focused on the patient.

In some embodiments, more than one measured physiological parameter may be displayed at once, for example two or measurements relating to anaesthesia. Thus, both a depth of consciousness measurement which is independent of evoked potentials and a depth of consciousness measurement which is based on the analysis of auditory evoked potentials may be displayed at the same time. For example, the number of adjacent light sources which are illuminated in a first outer circle of light sources and the number of adjacent light sources which are illuminated in a second inner circle of light sources may each depend on a respective measurement of a parameter relating to anaesthesia.

Additional sensors may be provided to measure data such as blood pressure, blood oxygen concentration, heart rate or a concentration of one or more molecules, for example specific hormones in the blood stream. Additional sensors may be integral with the headset or other wearable monitor, although they may be remote sensors which communicate measured physiological parameters to the headset or other wearable monitors, through wired or wireless connections.

The precise shape of the arms and the overall configuration of the headset may be adapted for particular applications. With reference to FIG. 5, the arms may include raised portions, for example, directly over electrodes, or between longitudinally spaced electrodes. The raised portions provide additional convenient gripping points, facilitating manipulation of the shape of the arms.

With reference to FIGS. 6A through 6E, the arms of the headset may have any of a number of different cross-sections, for example they may be rounded, or triangular, and may comprise a longitudinally extending flange to facilitate pinching. Typically, the cross-section is selected to enable the arms to be conveniently held and manipulated by pinching between the thumb and first finger, preferably whilst the thumb and first finger are in contact.

The arms are made from a compliant flexible material, such as a plastics material, or a silicone based material. The material may be resilient or may not hold its shape when flexed. The material is selected so that the arms may curve with a radius of curvature of not more than 10 cm, and preferably less, in the plane of the head-engaging surface, without buckling. Thus, they may be better adjusted to the shape of a patient's head than broad, planar sheets of adhesive material.

The headset may be adhered to the patient in a number of different ways. For example, in alternative embodiments, a small amount of adhesive may be provided along the lateral edge of the electrode. In this case each electrode typically had a breadth of at least 75% and more typically at least 90% of the breadth of the head-engaging surface of the respective flexible arm.

The arms can include a plurality of chambers having flexible walls and channels extending from the chambers to the head-engaging surface. Thus, when the arms are pinched, air is displaced from the chambers which can then be fitted to a patient's head and released. The reduced pressure within the chambers provides a sucking force retaining the arm in place. This may be sufficient in itself to retain the arms and thereby the electrodes in position. However, this adhesive mechanism may be combined with adhesive strips to provide improved long terms adhesion.

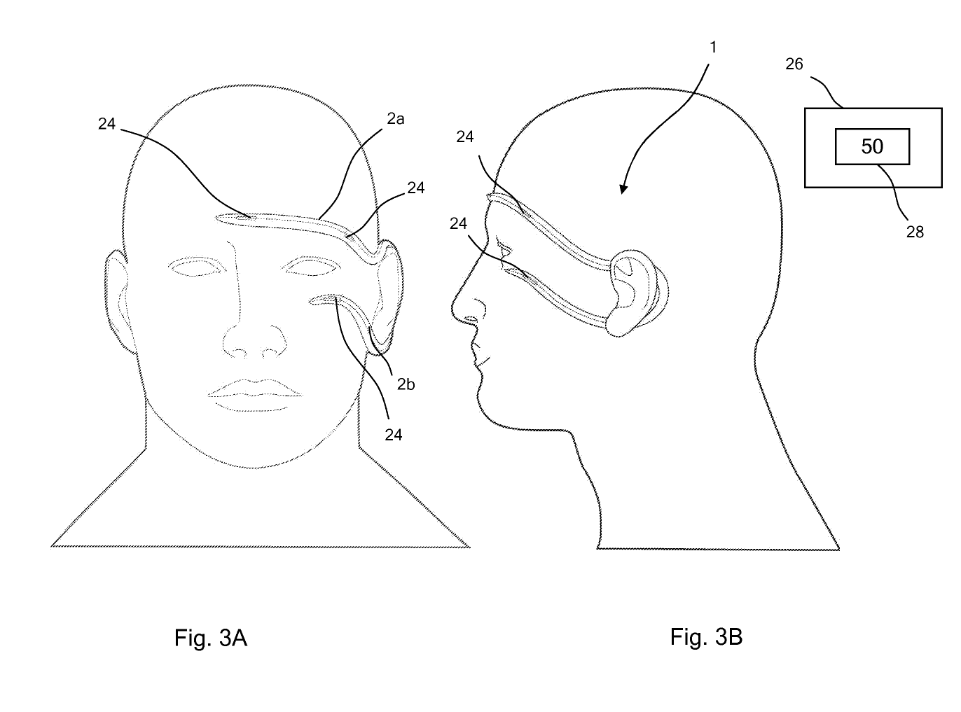

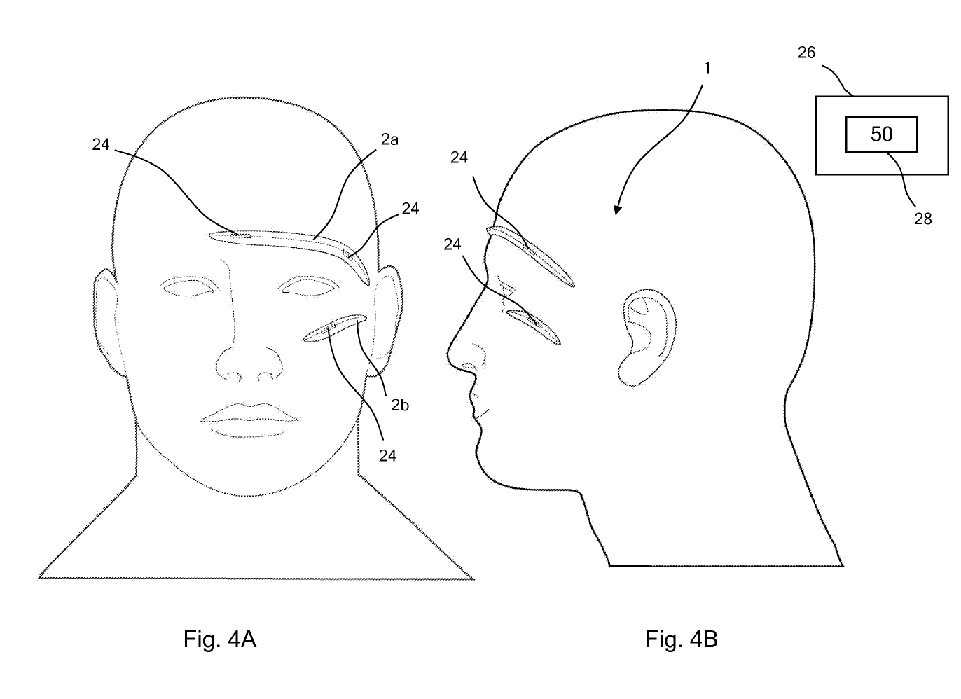

Although the example embodiment concerns a headset which includes a loudspeaker, for generating AEPs, the headset may also be provided without an earpiece, as illustrated in FIGS. 3A and 3B. The headset may lack a connection portion and the invention also encompasses an electrode support having a single compliant elongate support member or, as illustrated in FIGS. 4A and 4B, two electrode supports, each of which retains one or more electrodes may be used in combination. Two or more electrode supports may communicate with a remote monitoring device using a wired or wireless connection, or they may communicate with each other.

In alternative embodiments a greater or lesser number of longitudinally spaced electrodes may be provided support by a single compliant elongate support member. For example, three, or four, or more than four longitudinally spaced electrodes may be provided on the head-engaging surface of a single elongate support member, for example, for location on a patient's forehead in use.

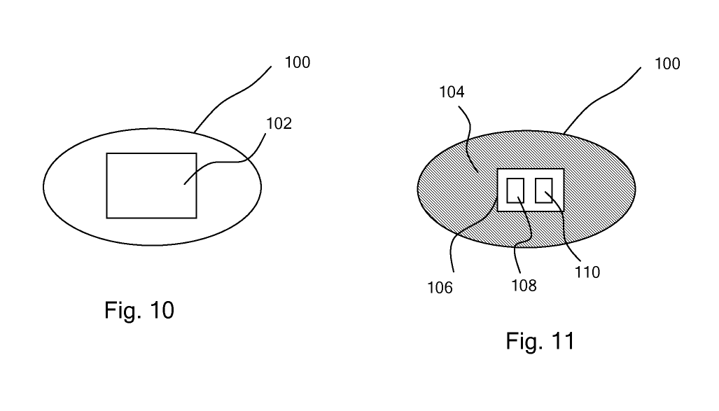

A wearable monitor having integral display means may be useful in many contexts other than a headset. For example, wearable monitors may be applied to other parts of the body and measure any of a large number of different physiological parameters which it is useful to display in situ, on the wearable monitor. For example, with reference to FIGS. 10 and 11 a wearable monitor may be formed as an adhesive patch 100, having a sensing surface 106 surrounded at least in part by an adhesive skin engaging surface 104 facing the patient's skin in use and an integral display 102 on the opposite outward surface of the adhesive patch. The sensing surface may comprise for example comprise an optical source 108 and optical detector 110 functioning as an oximeter to measure blood oxygen concentration or the concentration of one or more salts or other molecules, heart rate, or any other measurable physiological parameter. The physiological parameter, or a parameter derived therefrom, may be represented on the integral display as a numerical value, graph, chart, icon or in any other way.

Wearable monitors may have integral power supplies (for example, integral batteries) or scavenging power supplies (for example which obtain power from ambient heat or electromagnetic fields) and may be formed from flexible components such as flexible membrane, plastic electronic circuits, flexible sensing surfaces and flexible batteries.

Further variations and modifications may be made within the scope of the invention herein disclosed.

1. A wearable monitor comprising one or more fixing elements for fixing the wearable monitor to a body, a body-facing surface and a transducer for either or both stimulating a physiological response and measuring a physiological parameter, and display means or audio output means integral to the wearable monitor for displaying a signal related to a measured physiological parameter on the wearable monitor or transmitting a sound related to a measured physiological parameter from the wearable monitor, respectively.

2. A wearable monitor according to claim 1, wherein the body-facing surface has the transducer thereon.

3. A wearable monitor according to claim 1, wherein the body-facing surface is a skin-facing surface.

4. A wearable monitor according to a claim 1, wherein the one or more fixing elements comprise one or more adhesion means.

5. A wearable monitor according to claim 1, wherein the display means or audio output means are display means for displaying a signal or transmitting a sound related to a measured physiological parameter on the wearable monitor.

6. A wearable monitor according to claim 1, wherein the physiological parameter is a parameter specific to a part of the body to which the wearable monitor can be attached.

7. A wearable monitor according to claim 1, wherein the wearable monitor is a wearable sensor and the transducer is a sensor.

8. A wearable monitor according to claim 1, wherein the transducer comprises an actuator.

9. A wearable monitor according to claim 1, wherein the display means do not protrude significantly from the wearable monitor.

10. A wearable monitor according to claim 1, wherein the wearable monitor has a body engaging surface and an opposite outer surface and the display means are flush with the outer surface.

11. A wearable monitor according to claim 1, wherein the display means displays a visual signal dependent on the quality of the electrical connection between an electrode and body.

12. A wearable monitor according to claim 11, wherein the wearable monitor includes one or more electrodes and the visual indicator operable to display a visual indication dependent on the quality of the electrical connection between an electrode and body is located on an outer surface of the wearable monitor, directly over the respective electrode.

13. A wearable monitor according to claim 1, wherein the display means comprises one or more light sources operable to display a visual signal related to a measured physiological parameter by the intensity or colour of light emitted by the light source.

14. A wearable monitor according to claim 1, wherein the display means is operable to display an image including numbers or text related to a measured physiological parameter.

15. A wearable monitor according to claim 1, wherein the display means comprises a minimum display area of at least 8 cm².

16. A wearable monitor according to claim 1, wherein the display means is operable to display visual signals related to more than one physiological parameter.

17. A wearable monitor according to claim 1, operable to communicate a measured signal to a remote processor and to receive a physiological parameter calculated taking into account the communicated measured signal, wherein. the display means displays a visual indication related to the received calculated physiological parameter.

18. A wearable monitor comprising a body portion and a retaining portion;
the retaining portion demountably retaining the body portion; the body portion comprising a display means or audio output means for displaying a signal related to a measured physiological parameter on the wearable monitor or transmitting a sound related to a measured physiological parameter from the wearable monitor, respectively; the retaining portion comprising one or more fixing elements to fix the wearable monitor to a body, a body-facing surface, and a transducer for either or both stimulating a physiological response and measuring a physiological parameter.

19. A kit for a wearable monitor, the kit comprising a body portion and a retaining portion, the retaining portion demountably retaining the body portion;
the body portion comprising the display means or audio output means for displaying a signal related to a measured physiological parameter on the wearable monitor or transmitting a sound related to a measured physiological parameter from the wearable monitor, respectively; the retaining portion comprising the one or more fixing elements to fix the wearable monitor to a body (generally the body of a mammal, typically a human being), a body-facing surface, and a transducer for either or both stimulating a physiological response and measuring a physiological parameter.

20. A wearable transducer mounting comprising a compliant elongate support member having a body engaging surface, one or more fixing elements to fix the body engaging surface to a wearer's body, and a plurality of transducers longitudinally spaced along the compliant elongate support member.

21. A transducer mounting according to claim 20, wherein the transducers are longitudinally spaced along the body engaging surface.

22. A transducer mounting according to claim 20, wherein the said transducers are electrodes.

23. A transducer mounting according to claim 20, wherein the transducer mounting is for mounting transducers to a wearer's head and the body engaging surface is a skin engaging surface.

24. A transducer mounting according to claim 20, wherein the body engaging surface of the compliant elongate support member is a head engaging surface, and the plurality of transducers are a plurality of electrodes longitudinally spaced on the head engaging surface.

25. A transducer mounting according to claim 20, wherein the one or more fixtures are adhering means.

26. A transducer mounting according to claim 25, wherein the adhering means comprises one or more adhesive strips.

27. A transducer mounting according to claim 25, wherein the adhering means extend longitudinally on either side of each transducer, but do not extend laterally of each transducer.

28. A transducer mounting according to claim 25, wherein the adhering means comprises one or more compressible chambers provided within the elongate support member and at least one aperture extending from each compressible chamber through the elongate support member to the skin engaging surface.

29. A transducer mounting according to claim 20, wherein each transducer has a breadth of at least 75% of the breadth of the or body engaging surface where the respective electrode is mounted.

30. A transducer mounting according to claim 20, wherein the breadth of the elongate support member in the region of each transducer is less than 20 mm.

31. A transducer mounting according to claim 20, wherein the elongate support member has a length which is greater than 10 times its maximum breadth.

32. A transducer mounting according to claim 20, wherein the depth of the transducer mounting in the region of each transducer is at least 2 mm.

33. A transducer mounting according to claim 20, wherein the elongate support member may have a breadth to thickness ratio of between 0.25 and 4.0, at least in the region of each transducer.

34. A transducer mounting according to claim 20, which is flexible around an axis normal to the skin engaging surface, with a radius of curvature of less than 10 cm.

35. A transducer mounting according to claim 20, having a three sided cross-section along the majority of its length.

36. A transducer mounting according to claim 20, wherein the elongate support member comprises one or more raised portion which extend further from the skin engaging surface than surrounding regions of the elongate support member.

37. A transducer mounting according to claim 20, formed to retain at least two electrodes on a wearer's forehead, above the eyeline, and a third electrode on a wearer's cheek, below the eyeline.

38. A transducer mounting according to claim 20 wherein the transducer mounting comprises a connecting member, a first compliant elongate support member extending from the connecting member having a skin engaging surface, adhering means to adhere the skin engaging surface to a wearer's head, and a plurality of transducers on the skin engaging surface of the first compliant elongate support, and a second compliant elongate support member extending from the connection member and having a skin engaging surface, adhering means to adhere the skin engaging surface to a wearer's head, and at least one transducer on the skin engaging surface of the second compliant elongate support, wherein the first and second compliant elongate support members extend from the connecting member at spaced apart locations.

39. A transducer mounting according to claim 20, wherein the connecting member comprise a sound generation module for generating sounds in a wearer's ear or a fitment for demountably attaching a sound generation module to the transducer mounting.

40. A transducer mounting according to claim 20, comprising a body portion and a retaining portion demountably retaining the body portion; the retaining portion comprising the compliant elongate support member, the one or more fixing elements and a plurality of transducers longitudinally spaced along the compliant elongate support member, the body portion comprising a display means or audio output means for displaying a signal or transmitting a sound related to a physiological parameter measured by the transducers.

41. A kit for a transducer mounting, the kit comprising a body portion and a retaining portion configured to demountably retain the body portion; the retaining portion comprising the compliant elongate support member, adhering means and a plurality of transducers longitudinally spaced along the compliant elongate support member, the body portion comprising a display means or audio output means for displaying a signal or transmitting a sound related to a physiological parameter measured by the transducers.
